(19) United States
(12) Patent Application Publication
(10) Pub. No.: US 2012/0214843 A1
(43) Pub. Date: Aug. 23, 2012

(54) Combination Treatment of Hydroxpyridonate Actinide/Lanthanide Decorporation Agents

(75) Inventors: Patricia W. Durbin-Heavey, Oakland, CA (US); Leonore Frances Heavey, Baton Rouge, LA (US); Kenneth N. Raymond, Berkeley, CA (US); Rebecca J. Abergel, Berkeley, CA (US); David K. Shuh, Oakland, CA (US)

(73) Assignee: THE REGENTS OF THE UNIVERSITY OF CALIFORNIA, Oakland, CA (US)

(21) Appl. No.: 13/291,383

(22) Filed: Nov. 8, 2011

Related U.S. Application Data
(63) Continuation of application No. PCT/US2010/034266, filed on May 10, 2010.
(60) Provisional application No. 61/176,866, filed on May 8, 2009.

(51) Int. Cl. A61K 31/444; A61P 39/04

(52) U.S. Cl. 514/335

(57) ABSTRACT

The invention provides for a method for treating a subject in need of such treatment comprising administering a therapeutically effective amount of one or more pharmaceutical compositions comprising a 1,2-HOPO chelating agent and a 3,2-HOPO chelating agent to a subject in need of such treatment. The use of both 1,2-HOPO and a 3,2-HOPO chelating agents in combination is more effective than using only one chelating agent alone. The invention is especially useful when practiced on a subject that has been exposed to, have been in contact with, or contaminated by one or more known or unknown actinides and/or lanthanides, or a mixture thereof.

### RELATED PATENT APPLICATIONS

The application claims priority as a continuation application to PCT International Patent Application No. PCT/US2010/34266, filed May 10, 2010, which claims priority to U.S. Provisional Patent Application Ser. No. 61/176,866, filed May 8, 2009, which are herein incorporated by reference in their entireties.

### STATEMENT OF GOVERNMENTAL SUPPORT

The invention was made with government support under Contract No. DE-AC02-05CH11231 awarded by the U.S. Department of Energy and Grant No. 1R01A1074065-01 awarded by the National Institutes of Health. The government has certain rights in the invention.

### FIELD OF THE INVENTION

This invention relates generally to genetic markers involved in the treatment of radionuclide poisoning.

### BACKGROUND OF THE INVENTION

Exposure to radionuclides accidentally or deliberately scattered by a radiological dispersion device or deposited from a nuclear power plant accident or nuclear device detonation could result in the contamination of a large population. As internalized radionuclides are highly toxic and may cause both acute and chronic radiation injury, such contamination event would have dramatic public health consequences.

Decorporation by chelating agents is the only way to reduce exposure of certain incorporated isotope, and diethylenetriaminepentaacetic acid (DTPA) has been the standard therapy for actinide/lanthanide decorporation since its development and use by the U.S. Atomic Energy Commission in the 1950's. Unfortunately, the decorporation efficacy of DTPA is limited to transuranic radionuclides (e.g. plutonium, americium and curium) and it must be administered intravenously or by nebulizer, which would make administration in mass casualty situations challenging. Therefore, new practical radionuclide decorporation agents are greatly needed, as emphasized by several U.S. governmental agencies.

### SUMMARY OF THE INVENTION

The invention provides for a method for treating a subject in need of such treatment comprising administering a therapeutically effective amount of one or more pharmaceutical compositions comprising a 1,2-HOPO chelating agent and a 3,2-HOPO chelating agent to a subject in need of such treatment. The use of both 1,2-HOPO and a 3,2-HOPO chelating agents in combination is more effective than using only one chelating agent alone. The invention is especially useful when practiced on a subject that has been exposed to, have been in contact with, or contaminated by one or more known or unknown actinides and/or lanthanides, or a mixture thereof. Such subjects include those subjected to or exposed to an explosion caused by a “dirty bomb” or radiological dispersal device (RDD).

In some embodiments of the invention, the administration step comprises administering the 1,2-HOPO and 3,2-HOPO chelating agents simultaneously or at different times. When the 1,2-HOPO and 3,2-HOPO chelating agents are administered simultaneously, they can be administered in the same or separate pharmaceutical compositions.

In some embodiments of the invention, the subject is in need of such treatment because the subject is to be exposed, has been exposed, or is continuously exposed to one of more actinide and/or lanthanide, or a mixture thereof. In some embodiments of the invention, the subject is in need of such treatment because the subject is to come in contact with, was in contact with, or is continuously in contact to one of more actinide and/or lanthanide, or a mixture thereof. In some embodiments of the invention, the subject is in need of such treatment because the subject had ingested, will ingest, or is ingesting one of more actinide and/or lanthanide, or a mixture thereof. In some embodiments of the invention, the subject is in need of such treatment because the subject had breathed, will breath, or is breathing in one of more actinide and/or lanthanide, or a mixture thereof. The subject can be a human or non-human animal. The human can be a patient.

In some embodiments, the method further comprises administering to the subject a second pharmaceutical composition comprising one or more agents capable of chelating an actinide and/or lanthanide that is neither a 1,2-HOPO chelating agent nor a 3,2-HOPO chelating agent. Such agents are taught in Durbin, Health Physics 95(5): 465-492 (2008), hereby incorporated by reference.

The methods of the present invention are useful for decorporating, clearing or reducing the amount of actinide and/or lanthanide, or both from a subject. In some embodiments of the invention, the methods of the present invention are useful for decorporating, clearing or reducing the amount of actinide and/or lanthanide, or both from one or more systems or organs of the subject. In particular, the methods are useful for removing or reducing the amount of actinide and/or lanthanide, or both from the liver, kidney, soft tissue, and/or skeleton of the subject.

The present invention provides for a pharmaceutical composition comprising a 1,2-HOPO chelating agent, a 3,2-HOPO chelating agent, and a pharmaceutically acceptable carrier.

The present invention provides for the use of a 1,2-HOPO chelating agent and a 3,2-HOPO chelating agent in the manufacture of a medicant for use in the treatment of a subject that has been exposed to, have been in contact with, or contaminated by one or more known or unknown actinides and/or lanthanides, or a mixture thereof.

The present invention provides the use of two actinide and/or lanthanide chelating agents, the 1,2-HOPO chelating agent and 3,2-HOPO chelating agent, in combination as therapeutics for radionuclide decorporation. Both chelating agents are hydroxypyridinone derivatives that form stable complexes of actinide and/or lanthanide ions.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows the structures of 5-LIO(Me-3,2-HOPO) (“5LIO”) and 3,4,3-LI(1,2-HOPO) (“343LI”).

FIG. 2 shows the structure of diethylenetriamine pentaacetic acid (DTPA).

FIG. 3 shows the removal of ²³⁸Pu(IV) (Panel A) and ²⁴¹Am(III) (Panel B) by 5LIO, 343LI, and the combination of 5LIO and 343LI administered by intraperitoneal (ip) injection at 1 h and 24 h following introduction of ²³⁸Pu(IV) and ²⁴¹Am(III) into the mice. The combination treatment increased excretion of the actinides up to 5-8 times over the control.

FIG. 4 shows the removal of ²³⁸Pu(IV) (Panel A) and ²⁴¹Am(III) (Panel B) by 5LIO, 343LI, and the combination of 5LIO and 343LI administered by intraperitoneal (ip) injection at 1 h and 24 h following introduction of ²³⁸Pu(IV) and ²⁴¹Am(III) into the mice. The combination treatment promoted appreciable and significant reductions of liver and skeleton actinide.

FIG. 5 shows the removal of ²³⁷Np(V) (Panel A) and ²³³U(VI) (Panel B) by 5LIO, 343LI, and the combination of 5LIO and 343LI administered by intraperitoneal (ip) injection at 1 h and 24 h following introduction of ²³⁷Np(V) and ²³³U(VI) into the mice. The combination treatment increased excretion of the actinides up to 3 times over the control.

FIG. 6 shows the removal of ²³⁷Np(V) (Panel A) and ²³³U(VI) (Panel B) by 5LIO, 343LI, and the combination of 5LIO and 343LI administered by intraperitoneal (ip) injection at 1 h and 24 h following introduction of ²³⁷Np(V) and ²³³U(VI) into the mice. The combination treatment promoted appreciable and significant reductions of Np from liver Np and U from kidney.

FIG. 7 shows the removal of ²³⁸Pu(IV) (Panel A) and ²⁴¹Am(III) (Panel B) by 5LIO, 343LI, and the combination of 5LIO and 343LI orally administered at 1 h and 24 h following introduction of ²³⁸Pu(IV) and ²⁴¹Am(III) into the mice. The combination treatment increased excretion of the actinides up to 4-8 times over the control.

FIG. 8 shows the removal of ²³⁸Pu(IV) (Panel A) and ²⁴¹Am(III) (Panel B) by 5LIO, 343LI, and the combination of 5LIO and 343LI orally administered at 1 h and 24 h following introduction of ²³⁸Pu(IV) and ²⁴¹Am(III) into the mice. The combination treatment promoted appreciable and significant reductions of actinides from the liver and skeleton.

FIG. 9 shows the removal of ²³⁷Np(V) (Panel A) and ²³³U(VI) (Panel B) by 5110, 343LI, and the combination of 5LIO and 343LI orally administered at 1 h and 24 h following introduction of ²³⁷Np(V) and ²³³U(VI) into the mice. When administered at 1 h the combination treatment increased excretion of the actinides up to about 115% times compared to the control. When administered at 24 h the combination treatment increased excretion of the actinides up to about 2-2.5 times compared to the control.

FIG. 10 shows the removal of ²³⁷Np(V) (Panel A) and ²³³U(VI) (Panel B) by 5LIO, 343LI, and the combination of 5LIO and 343LI orally administered at 1 h and 24 h following introduction of ²³⁷Np(V) and ²³³U(VI) into the mice. When administered at 1 h or 24 h the combination treatment promoted significant reductions of Np from the liver (25% or 50% of the control) and U from the kidney (50% or 70% of the control).

### DETAILED DESCRIPTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a chelating agent” includes a plurality of such chelating agents, and so forth.

“Therapeutically effective amount” means that amount of the chelating agents that elicit the biological or medicinal response in a tissue system, animal or human sought by a researcher, veterinarian, medical doctor or other clinician, which response includes alleviation of the symptoms of the disease or disorder being treated. The specific amount of chelating agents needed to elicit the biological or medicinal response will depend on a number of factors, including but not limited to the disease or disorder being treated, the chelating agents being administered, the method of administration, and the condition of the patient.

These and other objects, advantages, and features of the invention will become apparent to those persons skilled in the art upon reading the details of the invention as more fully described below.

### The 1,2-HOPO and 3,2-HOPO Chelating Agents

The 1,2-HOPO and 3,2-HOPO chelating agents suitable for use in the present invention are taught in U.S. Pat. Nos. 4,698,431 (“Hydroxypyridonate Chelating Agents”), 5,634,901 (“3-Hydroxy-2(1H)-pyridonate Chelating Agents”), and 5,892,029 (“3-Hydroxy-2(1H)-pyridonate Chelating Agents”), all of which are hereby incorporated by reference.

Suitable 1,2-HOPO chelating agent include, but are not limited to, molecules defined by the structure:

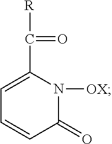

wherein R is a hydroxy group or

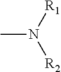

where R₁ and R₂ are selected from the group consisting of H, —CH₃, —CH₂CH₃ and —CH₂-φ, and X is either hydrogen, an alkali metal ion, or a quaternary ammonium ion.

Suitable 1,2-HOPO chelating agent include, but are not limited to, molecules incorporating a plurality of HOPO-type structures, including:

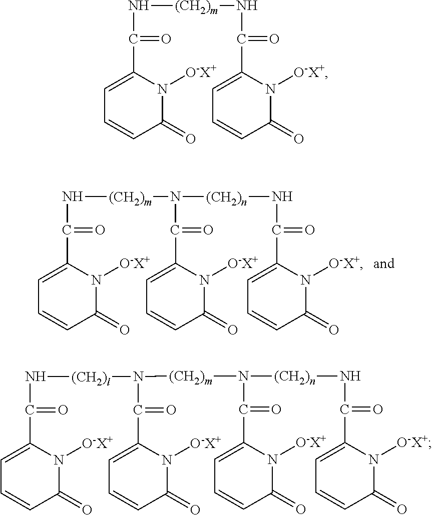

wherein l, m and n are integers between one and twenty. In a particular embodiment of the invention, m is three. In a particular embodiment of the invention, m is three and n is four. In a particular embodiment of the invention, 1 and n are three, and m is four

Suitable 1,2-HOPO and 3,2-HOPO chelating agents include, but are not limited to, a chelating agent comprised of a plurality of chelating functional units joined by one or more linking members, said chelating functional units independently selected from the group consisting of

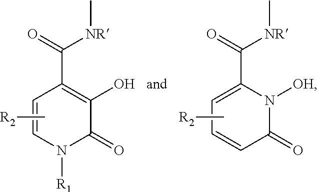

in which at least one of said plurality of chelating functional units on said chelating agent is

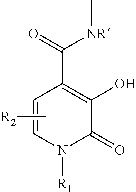

wherein R₁ and R₂ are independently selected from the group consisting of hydrogen, C₁₋₄ aliphatic hydrocarbon groups, and C₁₋₄ aliphatic hydrocarbon groups substituted by a single halide, hydroxy, carboxy, acrylamido group or an aryl group, and R′ is a member selected from the group consisting of a bond to a linking member, a hydrogen atom, C₁₋₈ aliphatic hydrocarbon groups, aryl groups, and C₁₋₈ aliphatic hydrocarbon groups substituted by amino, carboxy, or hydroxy groups.

Suitable 3,2-HOPO chelating agents include, but are not limited to, a chelating agent having the structure:

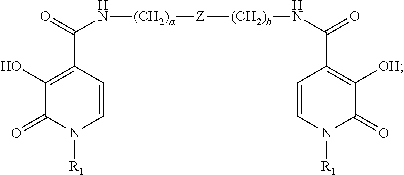

wherein R₁ is a member selected from the group consisting of hydrogen, C₁₋₄ aliphatic hydrocarbon groups, and C₁₋₄ aliphatic hydrocarbon groups substituted by a single halide, hydroxy, carboxy, or aryl group;
Z is a member selected from the group consisting of O, NH, N-alkyl, and N-aryl;
a is 2-4; and
b is 2-4.

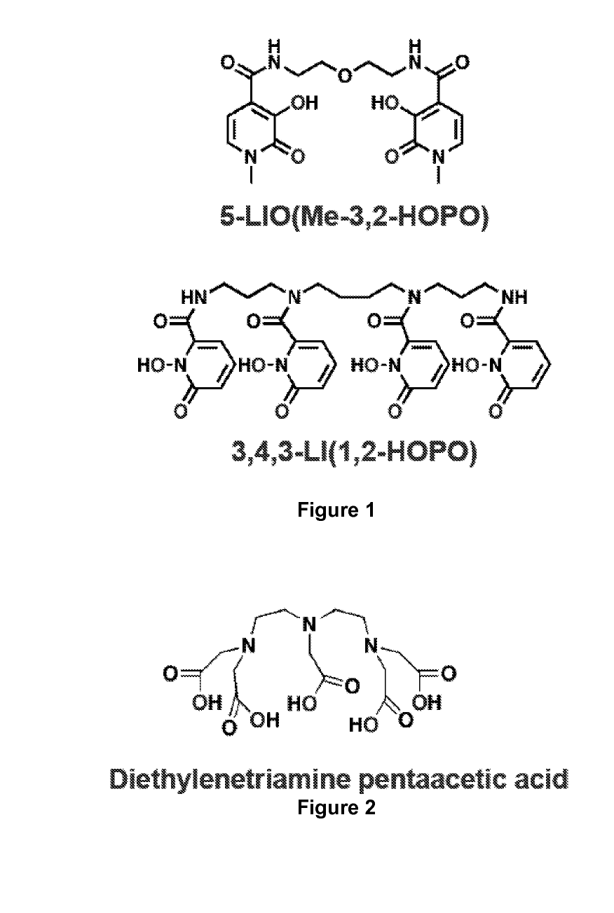

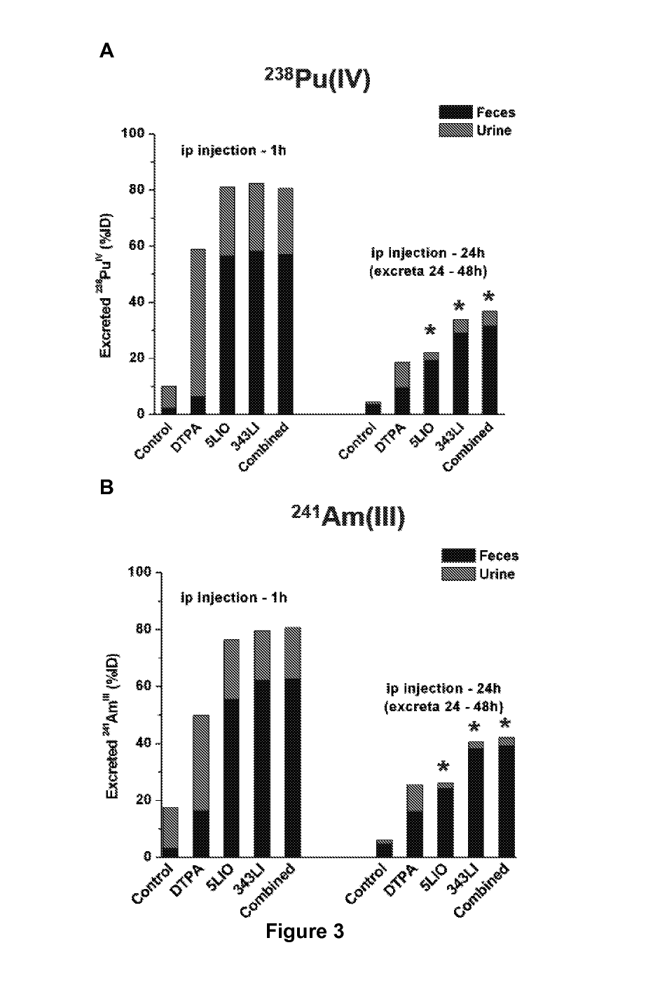

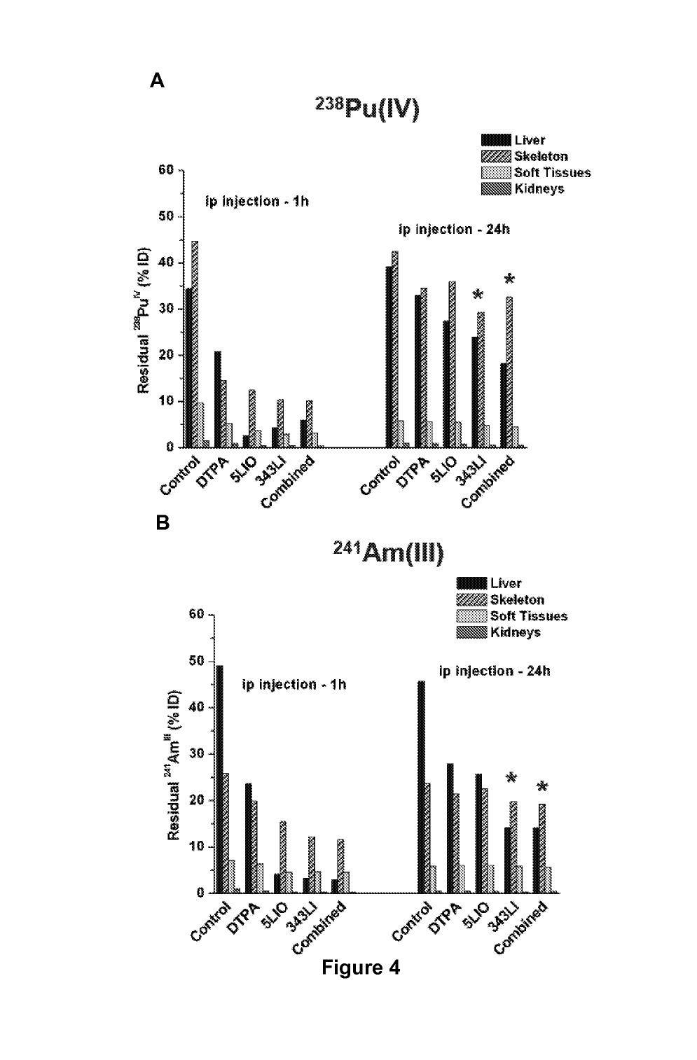

A suitable 1,2-HOPO and a suitable 3,2-HOPO are shown in FIG. 1.

The methods for synthesizing the 1,2-HOPO and 3,2-HOPO chelating agents are taught in U.S. Pat. Nos. 4,698,431; 5,634,901; and 5,892,029, all of which are hereby incorporated by reference.

The chelating agents are capable of binding or chelating, or capable of forming stable complexes with actinides and/or lanthanides, such as the cations of Eu, Pu, Np, Th, Am, and/or Cf, such as of ¹⁵²Eu(III), ²⁴¹Am(III), ²³⁸Pu(IV), ²³⁷Np(IV), ²³⁷Np(V), and ²³³U(VI).

The present invention includes within its scope prodrugs of the compounds of this invention. Such prodrugs are in general functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to a subject in need thereof. Conventional procedures for the selection and preparation of suitable prodrug derivatives are described, for example, in Wermuth, “Designing Prodrugs and Bioprecursors,” in Wermuth, ed., The Practice of Medicinal Chemistry, 2nd Ed., pp. 561-586 (Academic Press 2003). Prodrugs include esters that hydrolyze in vivo (for example in the human body) to produce a compound of this invention or a salt thereof. Suitable ester groups include, without limitation, those derived from pharmaceutically acceptable aliphatic carboxylic acids, particularly alkanoic, alkenoic, cycloalkanoic and alkanedioic acids, in which each alkyl or alkenyl moiety preferably has no more than six carbon atoms. Illustrative esters include formates, acetates, propionates, butyrates, acrylates, citrates, succinates, and ethylsuccinates.

### Modes of Administration and Pharmaceutical Formulations

Suitable modes of administration of the pharmaceutical composition include, but are not limited to, oral, topical, aerosol, inhalation by spray, parenteral, subcutaneous, intravenous, intramuscular, interperitoneal, rectal, and vaginal administration. The term parenteral, as used herein, includes subcutaneous injections, and intravenous, intrathecal, intramuscular, and intrasternal injection or infusion techniques. A particular mode of administration is one that brings a compound of this invention to the actual or potential site(s) of radionuclide contamination in the subject. The pharmaceutical composition can be in a solid, semi-solid, and/or liquid form.

The pharmaceutically acceptable carriers described herein, for example, vehicles, adjuvants, excipients, and diluents, are well known to those who are skilled in the art and are readily available. In some embodiments, the carrier is chemically inert to a compound of this invention and has no detrimental side effects or toxicity under the conditions of use. In some embodiments, the pharmaceutically acceptable carrier is free of pyrogen. The pharmaceutically acceptable carriers which can be used include, but are not limited to, water, glucose, lactose, gum acacia, gelatin, mannitol, starch paste, magnesium trisilicate, talc, corn starch, keratin, colloidal silica, potato starch, and urea.

The amount of the chelating agents that may be combined with the pharmaceutically acceptable carrier to produce a single dosage form will vary depending upon the subject treated and the particular mode of administration. Suitable dosage levels of the chelating agents include from about 1 mg to about 500 mg per kg body weight per day. In some embodiments, the suitable dosage level is from about 20 mg to about 100 mg per kg body weight per day. In some embodiments, the suitable dosage level is from about 10 μmol to about 100 μmol per kg body weight for 3,4,3-LI-1,2-HOPO. In some embodiments, the suitable dosage level is from about 30 μmol to about 200 μmol per kg body weight for 5-LIO-Me-3,2-HOPO. Dosage unit forms will generally contain from about 20 mg to about 100 mg of the chelating agents. In addition, the pharmaceutical composition can be administered on an intermittent basis, i.e., at daily, semi-weekly, or weekly intervals. It will be understood, however, that the specific dose level for a particular subject will depend on a variety of factors. These factors include the activity of the specific compound employed; the age, body weight, general health, sex, and diet of the subject; the time and route of administration and the rate of excretion of the chelating agents; the combination of chelating agents employed in the treatment; and, the severity of the particular disease or condition for which therapy is sought.

The pharmaceutical compositions suitable for oral administration include, but are not limited to, (a) liquid formulations; (b) capsules, sachets, tablets, lozenges, and troches, each containing a predetermined amount of the active ingredient, as solids or granules; (c) powders; (d) suspensions; and (e) suitable emulsions. Liquid formulations may include diluents, such as water and alcohols, and optionally a pharmaceutically acceptable surfactant. Capsule forms can be of the ordinary hard- or soft-shelled gelatin type containing, for example, surfactants, lubricants, and inert fillers. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and the like. The tablet can further comprise one or more colorants, diluents, buffering agents, disintegrating agents, moistening agents, preservatives, or flavoring agents.

The pharmaceutical composition, alone or in combination with other suitable components, can be made into aerosol formulations to be administered via inhalation. These aerosol formulations can be placed into pressurized acceptable propellants (such as dichlorodifluoromethane, propane, nitrogen, and the like) or non-pressured preparations (such as in a nebulizer or an atomizer). When the site(s) of infection of a subject is the lungs, a preferred mode of administration is inhalation of an aerosol formulation either orally or nasally. in particular, the aerosol formulation may comprises particles of a respirable size, including, but not limited to, mean particle sizes of 5 μm to 500 μm.

The pharmaceutical composition can be an injectable formulation. The requirements for effective carriers for injectable compositions are well known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)). In particular embodiments, injectable compositions are administered intravenously. Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The pharmaceutical composition can further comprise an excipient. Excipients that may be used include one or more carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference.

In vivo Efficacy. The octadentate 3,4,3-LI-1,2-HOPO is highly effective for Pu, Np, Th, Am and Cf chelation in vivo, and its efficacy greatly exceeds that of the current actinide chelation standard CaNa₃-DTPA at low dosage. For example, the efficiency of 3,4,3-LI-1,2-HOPO for clearing circulating Pu from mouse tissues ranges from 100 times (skeleton) to 240 times (soft tissues) that of CaNa₃-DTPA in five different protocols. In addition, the optimal activity dose of 3,4,3-LI-1,2-HOPO for removing newly deposited Pu from mice is 2.5% of the dose of CaNa₃-DTPA used clinically. The tetradentate 5-LIO-Me-3,2-HOPO has potential therapeutic value for Pu, U, Am and Np, and its efficiency for clearing circulating Pu from mouse tissues ranges from 5 times (skeleton) to 15 times (liver) that of CaNa₃-DTPA.

Oral Activity. Both compounds are orally active actinide chelators: when administered orally to mice or beagles after a Pu injection, 3,4,3-LI-1,2-HOPO and 5-LIO-Me-3,2-HOPO can remove up to 80% and 60%, respectively, of the injected Pu. In addition, pharmacokinetic studies using ¹⁴C-labeled ligands show that both compounds are stable to metabolic degradation and are significantly more effective than CaNa₃-DTPA for removing newly deposited Pu, Np, Am and U from mice.

Ligand Combination. Octadentate 3,4,3-LI(1,2-HOPO), HOPO(1), is highly effective for in vivo chelation of Pu(IV) and Am(III). Tetradentate 5-LIO(Me-3,2-HOPO), HOPO (2), is structurally suitable for chelating Np(V) and U(VI). Treatment is likely to be delayed in human contamination with dispersed radionuclides. Those conditions were approached, using mice, by ligand injection ip at 24 h or oral administration at 1 or 24 h after an iv actinide injection. Dosages of (1) and (2) were, respectively, 30 and 100 μmolkg⁻¹ ip and 100 and 200 μmolkg⁻¹ oral. Because mixtures of radionuclides may be released, (1) and (2) were combined to take advantage of their differing efficacies for the actinides. Dosages of combined (1) and (2) were, respectively, 30 plus 100 mmolkg⁻¹ injected ip and 100 and 200 mmolkg⁻¹ oral. Actinides in all mouse tissues and excreta were determined using published methods.

Injected at 24 h, both HOPOs and their mixture increased Pu(IV) and Am(III) excretion to 5-8 times control; appreciable and significant reductions of liver and skeleton actinide were obtained with (1) and the mixture. HOPO (2) and the mixture increased Np(V) and U(VI) excretion to about 3 times control, and significantly reduced liver Np(V) and kidney U(VI) to 30 and 45% control, respectively.

Given orally at 1 h, both HOPOs and their mixture increased Pu(IV) and Am(III) excretion to 4-7 times control, and significantly reduced both actinides in liver and bone. HOPO (2) and the mixture increased Np(V) and U(VI) excretion to about 115% control, and significantly reduced liver Np(V) and kidney U(VI) to 25% and 50% control, respectively. Oral treatment at 24 h with the HOPOs or their mixture increased excretion of Pu(IV) and Am(III) in 24 to 48 h to 4-8 times control; liver and body actinide were reduced significantly. HOPO (2) and the mixture increased 24-48 h post-treatment excretion of Np(V) and U(VI) to 2-2.5 times control, and significantly reduced liver Np(V) and kidney U(VI) to 50 and 70% control, respectively.

In all cases the combined ligands increased actinide excretion and reduced tissue actinide more (in most cases significantly more) than similar treatment with either ligand or CaNa₃-DTPA. Using the described hydroxypyridonate ligands as a combination therapy is a significant improvement for actinide decorporation purposes.

Octadentate 3,4,3-LI(1,2-HOPO), HOPO(1), is highly effective for in vivo chelation of Pu(IV) and Am(III). Tetradentate 5-LIO(Me-3,2-HOPO), HOPO (2), is structurally suitable for chelating Np(V) and U(VI). Treatment is likely to be delayed in human contamination with dispersed radionuclides. Those conditions were approached, using mice, by ligand injection ip at 24 h or oral administration at 1 or 24 h after an iv actinide injection. Dosages of (1) and (2) were, respectively, 30 and 100 μmolkg-1 ip and 100 and 200 μmolkg-1 oral. Because mixtures of radionuclides may be released, (1) and (2) were combined to take advantage of their differing efficacies for the actinides. Dosages of combined (1) and (2) were, respectively, 30 plus 100 μmolkg-1 injected ip and 100 and 200 μmolkg-1 oral. Actinides in all mouse tissues and excreta were determined using published methods.

Injected at 24 h, both HOPOs and their mixture increased Pu(IV) and Am(III) excretion to 5-8 times control; appreciable and significant reductions of liver and skeleton actinide were obtained with (1) and the mixture. HOPO (2) and the mixture increased Np(V) and U(VI) excretion to about 3 times control, and significantly reduced liver Np(V) and kidney U(VI) to 30 and 45% control, respectively.

Given orally at 1 h, both HOPOs and their mixture increased Pu(IV) and Am(III) excretion to 4-7 times control, and significantly reduced both actinides in liver and bone. HOPO (2) and the mixture increased Np(V) and U(VI) excretion to about 115% control, and significantly reduced liver Np(V) and kidney U(VI) to 25% and 50% control, respectively. Oral treatment at 24 h with the HOPOs or their mixture increased excretion of Pu(IV) and Am(III) in 24 to 48 h to 4-8 times control; liver and body actinide were reduced significantly. HOPO (2) and the mixture increased 24-48 h post-treatment excretion of Np(V) and U(VI) to 2-2.5 times control, and significantly reduced liver Np(V) and kidney U(VI) to 50 and 70% control, respectively. In all cases the HOPOs alone or combined increased actinide excretion and reduced tissue actinide more (in most cases significantly more) than similar treatment with CaNa3-DTPA.

The invention having been described, the following examples are offered to illustrate the subject invention by way of illustration, not by way of limitation.

### Example 1

The following experiment was performed to assess the efficacy of the combination treatment of the present invention:

Objective: to assay the efficacy of the two ligands at promoting radionuclide excretion with prompt or delayed administration, as single or combined treatments, and to identify target organs and excretion pathways. The test system used Swiss-Weber mice (˜35 g). Actinide loading involves intravenous injection of An-citrate complex at t₀. Samples of later taken of the whole skeleton, liver, kidneys, bulk soft tissues, urine, and feces (Σ=100%). (See Table 1.)

TABLE 1

| | | | Chelator Dosage (μmol/kg) | | |
|---|---|---|---|---|---|
| | Treatment | Sacrifice | 3,4,3-LI | 5-LIO | |
| Protocol | Time | Time | (1,2-HOPO) | (Me-3,2-HOPO) | DTPA |
| Single ip | 1 h | 24 h | 30 | 100 | 30 |
| | 24 h | 48 h | 30 | 100 | 30 |
| Single Oral | 1 h | 24 h | 100 | 200 | 100 |
| | 24 h | 48 h | 100 | 200 | 100 |

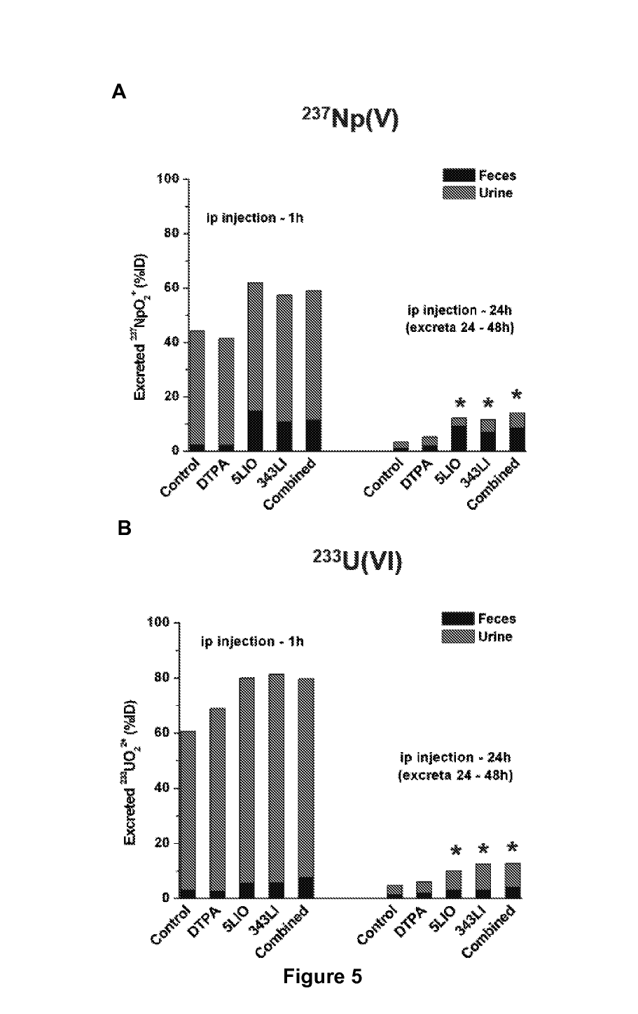

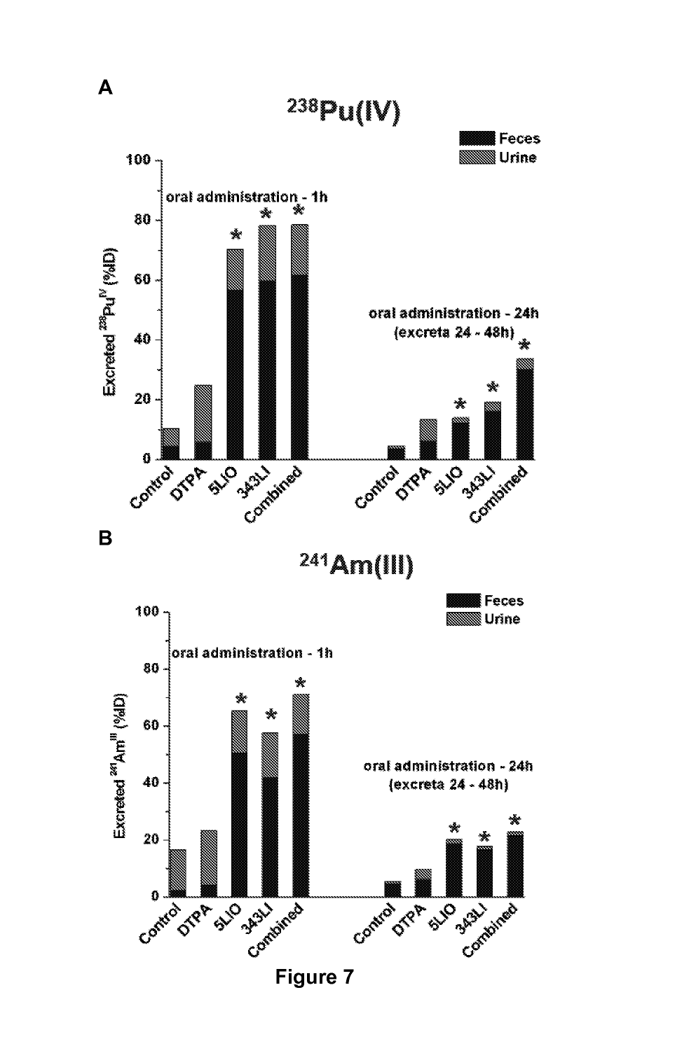

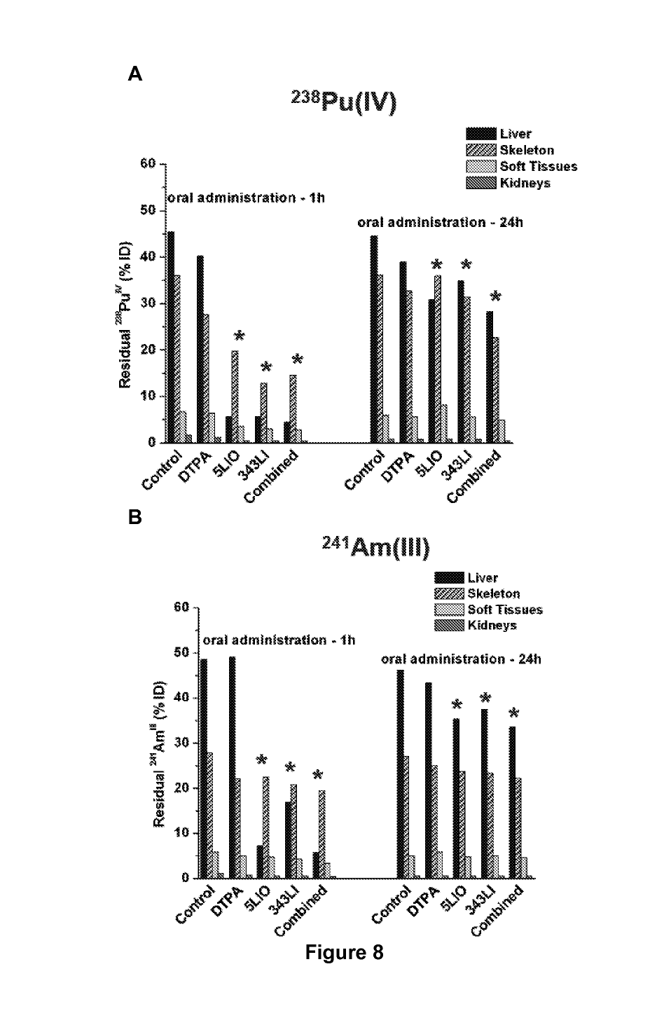

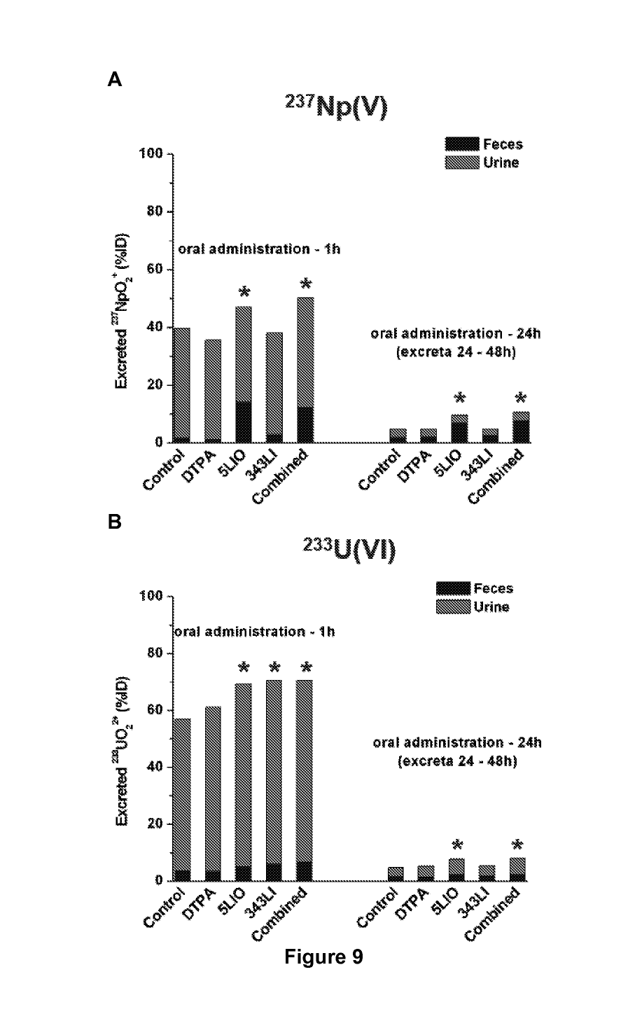

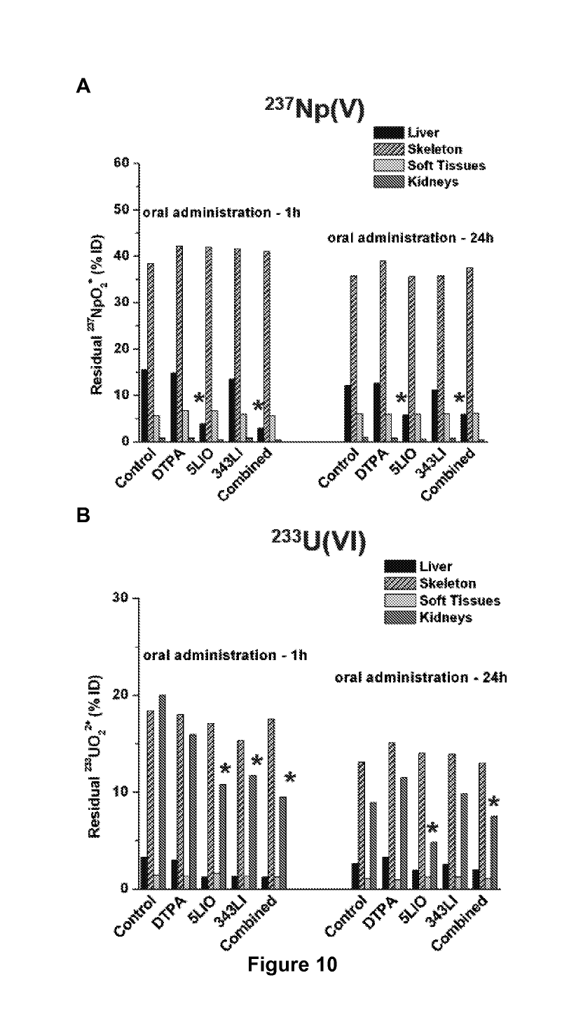

The results obtained are shown in FIGS. 5-10.

In all cases, the HOPO chelating agents in the combination treatment increased actinide excretion and reduced tissue actinide more than similar treatment with CaNa₃-DTPA or a single HOPO chelating agent alone. Both chelating agents chelate the actinides in the same body compartments. The combination significantly improves the overall results in the more demanding cases of oral or delayed administration.

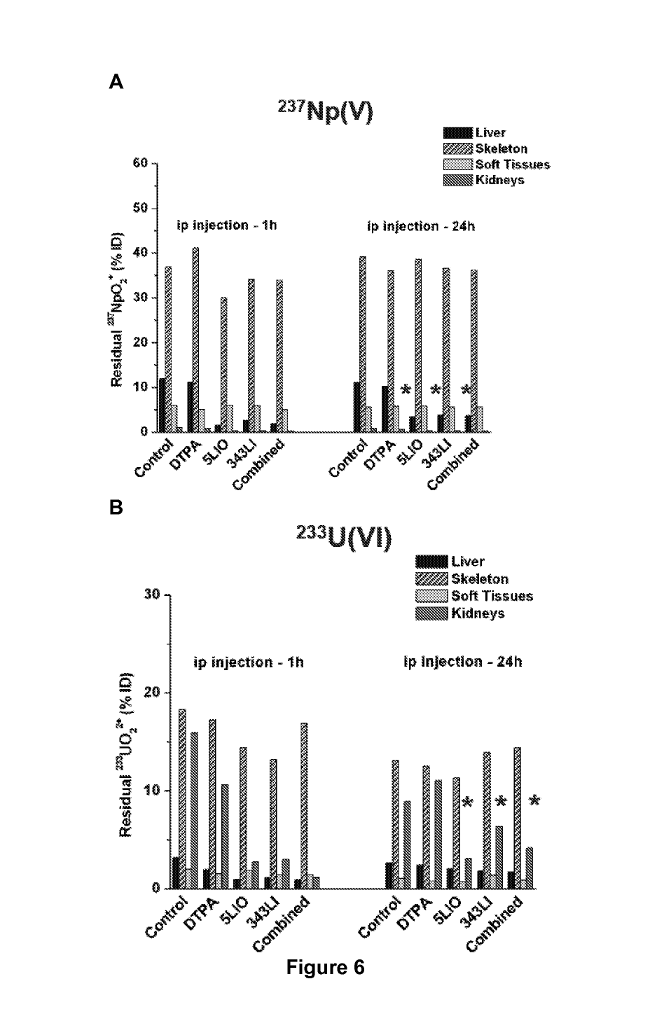

Prior to these results, it was understood that the dose dependence of the removal passed a certain concentration for each ligand (when used alone), and there is no decrease of residual metal (see FIG. 6 on page 474 of Durbin, Health Physics 95(5): 465-492 (2008)). This meant that if one injected twice the dose of each ligand alone, it will not improve the effect. However, these results reported herein demonstrate that if one administers both chelating agents the removal improves significantly, regardless of the metal to be removed. Moreover, on average, the pools from which the metals are chelated are the same for both ligands (usually skeleton/liver/kidney). Further, the magnitude of the synergistic effect is different when the combined 1,2-HOPO and a 3,2-HOPO chelating agents are injected or administered orally, at 1 h/24 h after metal injection.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

What is claimed is:

1. A method for treating a subject in need of such treatment comprising administering a therapeutically effective amount of one or more pharmaceutical compositions comprising a 1,2-HOPO chelating agent and a 3,2-HOPO chelating agent to a subject in need of such treatment.

2. The method of claim 1, wherein the subject has been exposed to, have been in contact with, or contaminated by one or more known or unknown actinides and/or lanthanides, or a mixture thereof.

3. The method of claim 2, wherein the subject has been subjected to or exposed to an explosion caused by a “dirty bomb” or radiological dispersal device (RDD).

4. The method of claim 1, wherein the administering step comprises administering the 1,2-HOPO and 3,2-HOPO chelating agents simultaneously.

5. The method of claim 1, wherein the administering step comprises administering the 1,2-HOPO and 3,2-HOPO chelating agents at different times.

6. The method of claim 4, wherein the 1,2-HOPO and 3,2-HOPO chelating agents are administered in the same pharmaceutical composition.

7. The method of claim 4, wherein the 1,2-HOPO and 3,2-HOPO chelating agents are administered in separate pharmaceutical compositions.

8. The method of claim 1, further comprising administering to the subject a second pharmaceutical composition comprising one or more agents capable of chelating an actinide and/or lanthanide that is neither a 1,2-HOPO chelating agent nor a 3,2-HOPO chelating agent.

9. The method of claim 1, wherein the administering step results in decorporating, clearing or reducing the amount of actinide and/or lanthanide, or both from the subject.

10. The method of claim 9, wherein the administering step results in decorporating, clearing or reducing the amount of actinide and/or lanthanide, or both from one or more systems or organs of the subject.

11. The method of claim 9, wherein the administering step results in removing or reducing the amount of actinide and/or lanthanide, or both from the liver, kidney, soft tissue, and/or skeleton of the subject.

12. The method of claim 1, wherein the 1,2-HOPO chelating agent is defined by the structure: 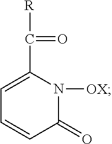
wherein R is a hydroxy group or 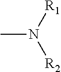
where R₁ and R₂ are selected from the group consisting of H, —CH₃, —CH₂CH₃ and —CH₂-φ, and X is either hydrogen, an alkali metal ion, or a quaternary ammonium ion.

13. The method of claim 12, wherein the 1,2-HOPO chelating agent is defined by one molecule selected from the group consisting 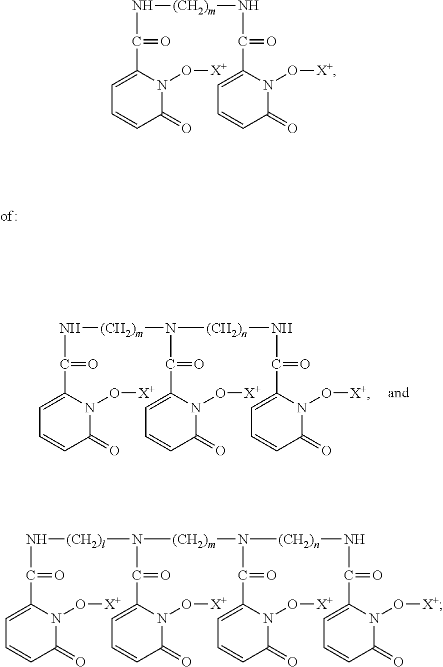
wherein l, m and n are integers between one and twenty.

14. The method of claim 13, wherein m is three.

15. The method of claim 14, wherein n is four.

16. The method of claim 13, wherein 1 and n are three, and m is four.

17. The method of claim 1, wherein the 1,2-HOPO chelating agent is 3,4,3-LI-1,2-HOPO.

18. The method of claim 2, wherein 1,2-HOPO chelating agent is 3,4,3-LI-1,2-HOPO and the actinides and/or lanthanides comprises a cation of Pu, Np, Th, Am or Cf.

19. The method of claim 1, wherein the 1,2-HOPO and 3,2-HOPO chelating agents comprise a plurality of chelating functional units joined by one or more linking members, said chelating functional units independently selected from the group consisting of 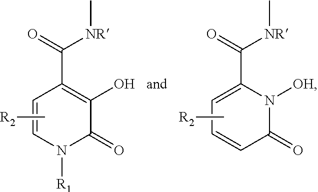
in which at least one of said plurality of chelating functional units on said chelating agent is 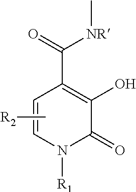
wherein R₁ and R₂ are independently selected from the group consisting of hydrogen, C₁₋₄ aliphatic hydrocarbon groups, and C₁₋₄ aliphatic hydrocarbon groups substituted by a single halide, hydroxy, carboxy, acrylamido group or an aryl group, and R′ is a member selected from the group consisting of a bond to a linking member, a hydrogen atom, C₁₋₈ aliphatic hydrocarbon groups, aryl groups, and C₁₋₈ aliphatic hydrocarbon groups substituted by amino, carboxy, or hydroxy groups.

20. The method of claim 1, wherein the 3,2-HOPO chelating agent is defined by the structure: 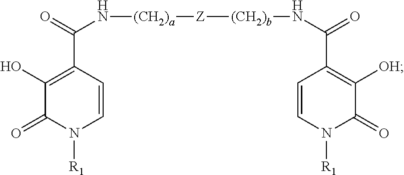
wherein R₁ is a member selected from the group consisting of hydrogen, C₁₋₄ aliphatic hydrocarbon groups, and C₁₋₄ aliphatic hydrocarbon groups substituted by a single halide, hydroxy, carboxy, or aryl group; Z is a member selected from the group consisting of O, NH, N-alkyl, and N-aryl; a is 2-4; and b is 2-4.

21. The method of claim 1, wherein the 3,2-HOPO chelating agent is 5-LIO-Me-3,2-HOPO.

22. The method of claim 21, the 1,2-HOPO chelating agent is 3,4,3-LI-1,2-HOPO.

23. The method of claim 2, wherein the 3,2-HOPO chelating agent is 5-LIO-Me-3,2-HOPO and the actinides and/or lanthanides comprises a cation of Pu, U, Am and Np.

24. The method of claim 2, wherein the actinides and/or lanthanides comprises a cation of Eu, Pu, Np, Th, Am, or Cf.

25. The method of claim 24, wherein the actinides and/or lanthanides comprises ¹⁵²Eu(III), ²⁴¹Am(III), ²³⁸Pu(IV), ²³⁷Np(IV), ²³⁷Np(V), or ²³³U(VI).

26. A pharmaceutical composition comprising a 1,2-HOPO chelating agent, a 3,2-HOPO chelating agent, and a pharmaceutically acceptable carrier.
